(19) United States
(12) Patent Application Publication
(10) Pub. No.: US 2016/0296552 A1
(43) Pub. Date: Oct. 13, 2016

(54) Treating Bacterial Infections of the Lung

(71) Applicants: Thomas Kennedy, Weston, FL (US); Cantex Pharmaceuticals, Inc., Weston, FL (US)

(72) Inventors: Thomas Kennedy, Charlotte, NC (US); Lin L. Mantell, East Meadow, NY (US)

(21) Appl. No.: 15/031,704

(22) PCT Filed: Oct. 23, 2014
(86) PCT No.: PCT/US14/62027

§ 371 Date: Apr. 22, 2016

Related U.S. Application Data
(60) Provisional application No. 61/895,324, filed on Oct. 24, 2013.
(60) Provisional application No. 61/901,286, filed on Nov. 7, 2013.

(51) Int. Cl. A61K 31/727; A61K 31/496; A61K 38/46; A61K 9/00; A61K 45/06

(52) U.S. Cl. CPC A61K 31/727; A61K 9/0019; A61K 9/007; A61K 45/06; A61K 38/465; A61K 31/496; C12Y 301/21001

(57) ABSTRACT

The present application provides methods of treating and preventing infections in the lung, such as Pseudomonas infections, comprising administering substantially non-anticoagulant 2-0, 2-0 desulfated heparin (ODSH) to subjects suffering from, or at risk for, chronic or acute pulmonary infections. ODSH can be administered alone or in combination with one or more other therapeutic agents, such as anti-microbial or antibiotic agents, mucolytic agents, DNases, bronchodilators, and anti-inflammatory agents. Also, provided herein are pharmaceutical compositions and unit dosage forms of ODSH, optionally in combination with other therapeutic agents, for use in the disclosed methods.

### 1. BACKGROUND

Pulmonary infection with bacteria and the subsequent lung inflammation to clear the invading pathogens significantly contributes to the morbidity and mortality of patients suffering from chronic or hospital-acquired pneumonia. Despite intensive antibiotic regimens and therapies targeted at treating the lung damage caused by the microbes and inflammation, antibiotic-resistant bacteria, especially Gram-negative bacteria, such as Pseudomonas aeruginosa, Burkholderia cepacia, Klebsiella pneumoniae, and Gram positive bacteria, such as Staphylococcus aureus, continue to be some of the most prevalent bacterial pathogens affecting many of these patients.

Pseudomonas is a ubiquitous, opportunistic pathogen, which causes many of the pulmonary infections with significant associated morbidity and mortality. Pseudomonas can cause pneumonia in patients with immunosuppression and chronic lung disease. Pseudomonas infection is of particular significance in subjects with other diseases affecting lung function, such as patients with cystic fibrosis (CF), chronic bronchitis, bronchiectasis, and chronic obstructive pulmonary disease (COPD). In CF patients, who have impaired mucociliary clearance of inhaled microbes, chronic infection of the lower respiratory tract with Pseudomonas aeruginosa (P. aeruginosa) is not only prevalent, it also contributes significantly to morbidity and mortality. Holby, 2011, BMC Medicine 9:32.

Bacterial infections of the lung can also be acquired nosocomially (in the hospital), for example in an intensive care unit (ICU) setting, especially where positive-pressure ventilation and/or endotracheal tubes are used. According to the United States Centers for Disease Control, the overall incidence of P. aeruginosa infections in U.S. hospitals averages about 0.4 percent (4 per 1000 discharges), and the bacterium is the fourth most commonly-isolated nosocomial pathogen, accounting for 10.1 percent of all hospital-acquired infections. In 2004, P. aeruginosa was reported to be the most common Gram negative bacterium in nosocomial infections. Berra et al., 2010, Minerva Anesthesiol. 76:824-832.

While a number of anti-bacterial agents targeting pathogens such as Pseudomonas are available, many of the bacteria exhibit significant ability to develop resistance to such agents. See Obritsch et al., 2005, Pharmacotherapy 25(10):1353-1364, reporting emergence of resistance in 27 to 72% of patients suffering from hospital-acquired infections that were initially responsive to therapy. In light of the high mortality and morbidity associated with Pseudomonas lung infections and the ability of the bacteria to develop resistance to antibiotic agents, non-antibiotic agents effective in treating bacterial lung infections are urgently needed.

### 2. SUMMARY

It has now been discovered that 2-O, 3-O desulfated heparin (ODSH), as a sole agent, reduces bacterial cell counts, acute lung injury, HMGB1 levels and HMGB1 binding to TLR2 and TLR4, and inflammatory cell infiltration in the lungs of mice infected with Pseudomonas aeruginosa, a model for pulmonary Pseudomonas infection. ODSH is also shown to improve survival of mice with Pseudomonas aeruginosa pneumonia. ODSH and compositions thereof are therefore useful in the treatment and/or prevention of Pseudomonas infections of the lung. Furthermore, as the mechanism by which ODSH acts appears to be unrelated to the particular type of bacterial pathogen causing the lung infection, ODSH and compositions thereof are equally useful in the treatment and/or prevention of infections of the lung caused by other bacterial pathogens.

In an aspect, the present disclosure provides a method of treating a pulmonary infection in a subject, such as a Pseudomonas infection. As described herein, the methods comprise administering to a subject suffering from a pulmonary infection, such as a Pseudomonas infection, a therapeutically effective amount of ODSH.

Pulmonary infection can be chronic or acute. Chronic infections occur in subjects who have an underlying condition impairing lung function or are immunosuppressed. A particular example of a condition impairing lung function is cystic fibrosis (CF). Other conditions associated with chronic infection are described below in the Detailed Description.

Acute pulmonary infections occur in particular settings or under circumstances that increase the risk of infection. In particular, acute infections occur in subjects who are hospitalized or live in group, e.g., nursing, homes, as well as subjects who require intubation, ventilation, or some other procedure in which a foreign object or material is introduced into the subject's airway. A particular example of an acute pulmonary Pseudomonas infection is ventilator-associated Pseudomonas infection. Acute infections can also occur in patients who are susceptible to chronic infection, such as Pseudomonas infection, as described herein.

Nosocomial pneumonia (NP, or hospital-acquired pneumonia) is associated with infections originating from hospital borne pathogens, in particular Pseudomona aeruginosa. Pseudomona aeruginosa pneumonia is characterized by excessive secretion of inflammatory cytokines, neutrophil infiltration, and subsequent lung damage. Persistent microbial presence and acute lung injuries are common features of these infections, contributing to high mortality rates.

Suitable subjects for treatment are those suffering from an acute or chronic pulmonary infection, such as a Pseudomonas infection, including subjects suffering from an underlying condition or disease, such as CF.

In an aspect, the present disclosure provides a method of preventing pulmonary infection such as Pseudomonas infection in a subject, comprising administering a prophylactically effective amount of ODSH to a subject at risk for acute or chronic pulmonary infection as described herein.

ODSH, or compositions thereof, may be administered alone, for a specified period of time or continuously.

Alternatively, ODSH may be administered in combination with, or adjunctive to, one or more other therapeutic agents. The one or more other therapeutic agents may be antibiotic or anti-microbial agents that also target the Pseudomonas infection and/or infections caused by other bacterial pathogens. In a specific embodiment, ODSH is administered in combination with tobramycin, aztreonam, ciprofloxacin, or levofloxacin.

Where the subject suffers from an underlying condition or disease that predisposes the subject to infection, the one or more other therapeutic agents may be agents that target a symptom associated with the underlying condition.

In some embodiments, the subject has cystic fibrosis and the one or more other therapeutic agents can include a mucolytic agent, a bronchodilator, and/or an anti-inflammatory agent. In a specific embodiment, the subject suffers from CF and ODSH is administered in combination with deoxyrinbonuclease I (DNase), e.g., dornase alfa.

ODSH and optional other therapeutic agents, or compositions thereof, may be administered parenterally or by inhalation. When administered parenterally, ODSH and optional other therapeutic agents can be administered by intravenous injection (e.g., bolus or infusion) or subcutaneous injection. When administered by inhalation, ODSH and optional other therapeutic agents can be administered as an aerosol by way of a nebulizer, a dry powder inhaler, a pressurized metered dose inhaler, or any other suitable device. When ODSH is administered in combination with another agent, it can be administered by the same or a different route.

Also provided herein are pharmaceutical compositions and unit dosage forms of ODSH, alone or in combination with other therapeutic agents, suitable for use in the methods described above. The pharmaceutical compositions may be formulated for administration according to any of the routes specified above, such as parenteral and by inhalation. Pharmaceutical compositions for parenteral administration can be suitable to dose ODSH at amounts ranging from about 1 mg/kg to about 50 mg/kg for bolus doses, or from about 0.1 mg/kg/hr to about 2.5 mg/kg/hr for infusions, or from about 25 mg to 800 mg in volumes of 2.0 mL or less per injection site, for subcutaneous injections. Pharmaceutical compositions for aerosol administration can be suitable to dose ODSH at amounts ranging from about 25 mg to about 800 mg.

Suitable unit dosage forms of ODSH include injectables and capsules for aqueous or powder aerosolization, as described in further detail below. In a specific exemplary embodiment, ODSH is formulated with dornase alfa in a unit dosage form suitable for inhalation. In another specific exemplary embodiment, ODSH is formulated with tobramycin, aztreonam, ciprofloxacin, or levofloxacin in a unit dosage form suitable for inhalation.

### 3. BRIEF DESCRIPTION OF THE FIGURES

FIG. 1 provides a bar chart of the number of viable bacteria, expressed on a logarithmic scale as the mean number of colony forming units (CFU) per milliliter of lung tissue recovered from mice inoculated with 5×10⁸ CFU of P. aeruginosa and treated at 5 minutes before and 12 hours after inoculation with subcutaneous administration of 0, 16.4, 49.3, or 148 mg/kg O-desulfated heparin (ODSH). (*) indicates that the measured mean viable cell count was statistically significant (p<0.05) when compared to the control group receiving no ODSH;

FIG. 2 provides a bar chart of the bacterial burden, expressed on a logarithmic scale as the mean number of colony forming units (CFU) per milliliter of lung homogenate recovered from mice inoculated with 5×10⁸ CFU of P. aeruginosa and treated at 0 hr and 12 hr after inoculation with subcutaneous administration of 0, 8.3, 25, or 75 mg/kg O-desulfated heparin (ODSH). (**) indicates that the measured value is significantly different compared to the control group (p≦0.01);

FIG. 3 provides a bar chart of the bacterial burden, expressed on a logarithmic scale as the mean number of colony forming units (CFU) per milliliter of bronchoalveolar lavage (BAL) recovered from mice inoculated with 5×10⁸ CFU of P. aeruginosa and treated at 0 hr and 12 hr after inoculation with subcutaneous administration of 0, 8.3, 25, or 75 mg/kg O-desulfated heparin (ODSH). (*) indicates that the measured value is significantly different compared to the control group (p≦0.05);

FIG. 4. provides a bar chart of the total protein content (μg/ml) in lung lavage fluid from mice inoculated with 5×10⁸ CFU of P. aeruginosa and treated at 5 minutes before and 12 hours after inoculation with subcutaneous administration of 0, 16.4, 49.3, or 148 mg/kg O-desulfated heparin (ODSH). (*) indicates that the measured protein content was statistically significant (p<0.05) when compared to the control group receiving no ODSH;

FIG. 5 provides a bar chart depicting the total protein content in BAL samples of control mice (not treated with ODSH), in which the BAL samples were isolated from the control mice at 0, 8, 16 and 24 hr post inoculation and the protein concentration is expressed as a percentage of total protein content in samples at 24 hr post-infection. (*) indicates that the measured value is significantly different compared to the value at 0 hr (*p≦0.05 and ***p≦0.001);

FIG. 6 provides a bar chart depicting total protein content in BAL samples from P. aeruginosa-infected mice treated with 0, 8.3, 25, or 75 mg/kg ODSH and presented as a percentage of the control group. (*) indicates that the measured value is significantly different compared to the control group (p≦0.05);

FIG. 7 depicts images at 10× magnification of right lungs of mice with P. aeruginosa infection treated with 0, 8.3, 25, or 75 mg/kg ODSH;

FIG. 8 provides a bar chart of the number of infiltrated cells (×10⁷/mL) in bronchoalveolar lavage fluid (BALF) from mice inoculated with 5×10⁸ CFU of P. aeruginosa and treated at 5 minutes before and 12 hours after inoculation with subcutaneous administration of 0, 16.4, 49.3, or 148 mg/kg O-desulfated heparin (ODSH); each value represents the mean (+/−standard error) of seven independent experiments with 17 to 23 mice per group;

FIG. 9 provides a bar chart of the number (×10⁶/ml) of neutrophils in lung lavage fluid from mice inoculated with 5×10⁸ CFU of P. aeruginosa and treated at 5 minutes before and 12 hours after inoculation with subcutaneous administration of 0, 16.4, 49.3, or 148 mg/kg O-desulfated heparin (ODSH); each value represents the mean (+/−standard error) of seven independent experiments with 17 to 23 mice per group;

FIG. 10 depicts the western blot analysis of HMGB1 levels in BAL samples isolated from control mice (not treated with ODSH) that were inoculated intratracheally with 0.5×10⁸ CFU of P. aeruginosa, and euthanized at different time points post-inoculation;

FIG. 11 depicts the western block analysis of HMGB1 levels in BAL samples isolated 24 hours post-inoculation with 5×10⁸ CFU of P. aeruginosa from mice treated with 0, 8.3, 25 or 75 mg/kg of ODSH;

FIG. 12 depicts a graph showing the effect of ODSH on HMGB1 binding to TLR2 in a competitive binding assay;

FIG. 13 depicts a graph showing the effect of ODSH on HMGB1 binding to TLR4 in a competitive binding assay; and

FIG. 14 depicts the survival post-inoculation of mice treated with either 75 mg/kg ODSH or saline every 12 hr.

### 4. DETAILED DESCRIPTION

It has been discovered, as described in detail in the Examples below, that an O-desulfated heparin (ODSH) that is low in anticoagulant activity is effective to treat Pseudomonas infections in the lung. Specifically, using an in vivo murine model of Pseudomonas lung infection, applicants have demonstrated that ODSH reduces bacterial load, recruitment of inflammatory cells, acute lung injury, HMGB1 levels, and inhibits binding of HMGB1 to TLR2 and TLR4 in the lungs of mice infected with Pseudomonas aeruginosa. In addition, ODSH improves survival of mice with Pseudomonas aeruginosa pneumonia. Furthermore, these effects were seen when ODSH is administered parenterally, via subcutaneous injection. Consequently, it is now appreciated that ODSH can be administered to treat Pseudomonas infections of the lung, and that administration can be either directly to the lungs via an aerosol or by a parenteral route, such as subcutaneous or intravenous. Thus, ODSH and compositions thereof suitable for aerosol or parenteral administration are useful in the treatment of Pseudomonas infections of the lung and in particular, Pseudomonas aeruginosa pneumonia.

Without intending to be bound by any theory of operation, the ability of ODSH to treat symptoms associated with bacterial lung infections operates by a mechanism that does not appear to be specific to the particular bacterial species causing the infection. Therefore, the methods of the present disclosure are generally applicable to bacteria commonly implicated in the chronic and acute lung infections described herein, including Gram negative bacteria (e.g., Pseudomonas aeruginosa, Burkholderia cepacia, Klebsiella pneumoniae) and Gram positive bacteria (e.g., Staphylococcus aureus).

While not being bound by any particular theory of mechanism, high mobility group box 1 (HMGB1), a recently discovered potent pro-inflammatory cytokine, appears to play a role in serious bacterial lung infection, such as from Pseudomonas aeruginosa, by compromising innate immunity by impairing phagocyte function through toll-like receptors (TLR) TLR4 and to a lesser extent TLR2. ODSH is shown here to decrease levels of airway HMGB 1 and blunt binding of HMGB 1 to receptors TLR2 and TLR4.

4.1. Methods of Treating and Preventing Pulmonary Pseudomonas Infections

In a first aspect, the present disclosure provides a method of treating a pulmonary infection in a subject, such as Pseudomonas, the method comprising administering a therapeutically effective amount of ODSH or other low-anticoagulating heparinoid to the subject.

The method can be used to treat chronic or acute pulmonary infections, such as but not limited to Pseudomonas infections. Chronic infections occur in subjects who are predisposed to, or at an increased risk of, infection, generally due to the presence of an underlying condition affecting lung function or immune system function. Chronic Pseudomonas pulmonary infections occur, for example, in subjects suffering from bronchiectasis of any origin, cystic fibrosis (CF), chronic obstructive pulmonary disease (COPD), asthma, emphysema, chronic bronchitis, acquired immune deficiency syndrome, or cancer.

Acute infections can occur in subjects, regardless of their predisposition to infection and generally occur in particular settings or under circumstances that increase the risk of infection. Settings include nursing or group homes, hospitals or particular units within hospitals, such as intensive or critical care units, in which subjects are at increased risk of acquiring an infection. Circumstances that can lead to acute pulmonary infection, such as Pseudomonas infection, generally involve the introduction of a foreign object or material into a subject's airway carrying, or providing entry for, a pathogen into the lungs. Endotracheal intubation, ventilation, and tracheostomy are examples of procedures that can lead to acute pulmonary infections, such as Pseudomonas infections. Hyperoxia may also predispose a subject to pulmonary infections. Hyperoxia is a condition, commonly attributed to ventilators, which involves excess oxygen in the lungs or other body tissues. Prolonged exposure to hyperoxia can lead to oxygen toxicity that increases a subject's susceptibility to infections. Oxygen toxicity is a major contributing factor to the development of ventilator associated pneumonia (VAP).

Nosocomial pneumonia (NP, or hospital-acquired pneumonia) is associated with infections originating from hospital borne pathogens, in particular Pseudomonas aeruginosa. Pseudomonas aeruginosa pneumonia is characterized by excessive secretion of inflammatory cytokines, neutrophil infiltration, and subsequent lung damage. Persistent microbial presence and acute lung injuries are common features of these infections, contributing to high mortality rates.

In certain embodiments, ODSH reduces pulmonary bacterial load (or burden) in subjects with lung infections. Bacterial load in the lungs of subjects treated with ODSH may be reduced by at least a factor of two, at least a factor of 3, at least a factor of 4, at least a factor of 5, at least a factor of 6, at least a factor of 7, at least a factor of 8, at least a factor of 10 at least a factor of 11 or at least a factor of 12 relative to the bacterial load of the subject prior to treatment with ODSH. The bacterial load may be determined, for example, using procedures described herein in the Examples and in Entezari et al., 2012, Mol. Med., 18:477-485 and Patel et al., 2013, Am. J. Respir. Cell Mol. Biol. 48:280-287, the disclosures of which are incorporated herein in their entirety by reference. In certain embodiments, the subject has Pseudomonas aeruginosa pneumonia and the bacterial load is reduced by at least a factor of 5 relative to the bacterial load prior to treatment with ODSH.

ODSH may ameliorate lung injuries in subjects with lung infections. Lung injury may be characterized by increased total protein content in airways. The extent of lung injury in a subject may be determined by measuring the protein content in the lungs, for example, using procedures described herein in the Examples and in Patel et al., 2013, Am. J. Respir. Cell Mol. Biol. 48:280-287. Subjects treated with ODSH may display a reduction in protein content in the lungs of at least 10%, at least 20%, at least 30%, at least 40%, at least 50%, at least 60%, at least 70%, at least 80% or at least 90%, relative to protein content in the lungs prior to treatment with ODSH.

Lung injury may be characterized by cell infiltration in airways. The extent of lung injury in a subject may be determined by measuring the total cell count in the lungs, for example, using procedures described herein in the Examples and standard hemocytometer procedures for determining cell counts. Subjects treated with ODSH may display a reduction in total cell count in the lungs of at least 10%, at least 20%, at least 30%, at least 40%, at least 50%, at least 60%, at least 70%, at least 80% or at least 90%, relative to total cell count in the lungs prior to treatment with ODSH. Subjects treated with ODSH may display a reduction in neutrophil count in the lungs of at least 10%, at least 20%, at least 30%, at least 40%, at least 50%, at least 60%, at least 70%, at least 80% or at least 90%, relative to neutrophil count in the lungs prior to treatment with ODSH.

ODSH may reduce infection-induced elevation of HMGB1 in subjects with lung infections. HMGB 1 content in the lungs may be determined, for example, using procedures described herein in the Examples and in Patel et al., 2013, Am. J. Respir. Cell Mol. Biol. 48:280-287. Subjects treated with ODSH may display a reduction in HMGB1 in the lungs of at least 10%, at least 20%, at least 30%, at least 40%, at least 50%, at least 60%, at least 70%, at least 80% or at least 90%, relative to HMGB1 in the lungs prior to treatment with ODSH. In certain embodiments, ODSH may decrease binding of HMGB1 to TLR2 and/or TLR4.

ODSH may improve survival of subjects with lung infections. A subject suffering from a pulmonary infection may show enhanced innate immunity and/or ameliorated lung injury when treated with ODSH. ODSH may rescue or ameliorate hyperoxia-compromised innate immunity. In particular, ODSH may improve survival of subjects with pneumonia such as Pseudomona aeruginosa pneumonia.

Suitable subjects are those suffering from an infection of the lungs, such as a Pseudomonas infection, whether chronic or acute, as described above. Subjects in whom chronic pulmonary infection, such as a Pseudomonas infection, is common include subjects suffering from a condition affecting lung function or immune system function including, by way of example but not limitation, bronchiectasis of any origin, cystic fibrosis, chronic obstructive pulmonary disease, asthma, emphysema, pneumonia, chronic bronchitis, acquired immune deficiency syndrome, and/or cancer.

Subjects in whom acute pulmonary infection, such as a Pseudomonas infection, can occur are those in an environment in which there is an increased risk of contracting an infection as well as individuals subjected to procedures that increase the risk of introducing the pathogen into the lungs. Subjects include, by way of example and not limitation, subjects who are hospitalized, in an intensive or critical care unit, or at risk of contracting a nosocomial infection (e.g., hospital workers), and subjects who are or have been intubated or on a ventilator. Included herein are also subjects who are susceptible to chronic pulmonary infection, such as a Pseudomonas infection, as described above.

The present disclosure also provides a method of preventing a pulmonary infection, such as a Pseudomonas infection, in a subject, comprising administering a prophylactically effective amount of ODSH to a subject at risk of developing an infection, such as a Pseudomonas infection. Suitable subjects are subjects who do not currently have a pulmonary infection, such as a Pseudomonas infection, but are at risk of developing a chronic or acute infection, as described above.

The methods of the present disclosure can be used to treat and/or provide prophylaxis for a broad range of subjects. A suitable subject for receiving treatment and/or prophylaxis as described herein is any mammalian subject in need thereof, in particular a human patient. Examples of human subjects include, but are not limited to, pediatric patients, adult patients, and geriatric patients.

It may be desirable to administer ODSH in combination with one or more additional therapeutic agents. Additional therapeutic agents may target a pulmonary infection, such as Pseudomonas infection, or may target some aspect of lung function that is compromised in the specific subject to be treated. The specific agent(s) administered in combination with ODSH will depend on the subject being treated.

In some instances, it may be desirable to combine ODSH with therapeutic agents that target Pseudomonas and/or other bacterial pathogens. In such cases, ODSH can be administered in combination with an anti-microbial or antibiotic agent. Suitable anti-microbial or antibiotic agents include, but are not limited to, aminoglycosides (e.g., gentamicin, amikacin, tobramycin), quinolones or fluoroquinolones (e.g., ciprofloxacin, ciproflaxin betaine, levofloxacin, and moxifloxacin), cephalosporins (e.g., ceftazidime, cefepime, cefoperazone, cefpirome), antipseudomonal penicillins and/or β-lactams (including ureidopenicillins and carboxypenicillins, piperacillin, piperacillin-tazobactam, ticarcillin, ticarcillin-clavulanate, mezlocillin, and azlocillin), carbapenems (e.g., meropenem, imipenem, doripenem), polymyxins (such as polymyxin B and colistin), macrolides, glycylcycline antibiotics such as tigecycline, glycopeptides antibiotic compounds, and monobactams (such as aztreonam).

In some embodiments, ODSH and antibiotic or anti-microbial agents can be administered parenterally. In some embodiments, antibiotic or anti-microbial agents, for example, but not limited to, aztreonam, tobramycin, and fluoroquinolones (e.g., ciprofloxacin), are administered by inhalation. In a specific example, ODSH and tobramycin are administered by inhalation. Patients receive a 300 mg nominal dose tobramycin, administered as an aerosol of a 5 ml dose with a standard jet nebulizer twice daily, on a 28 day “on” therapy followed by a 28 day “off” period, to reduce the potential for development of resistant bacterial strains. See, also, U.S. Pat. No. 5,508,269, the disclosure of which is incorporated herein in its entirety by reference.

Many of the subjects at risk for and suffering from pulmonary infection, such as a Pseudomonas infection, have pre-existing conditions affecting lung function and/or immune function that predispose them to bacterial infection. Other therapeutic agents may be administered adjunctive to, or in combination with ODSH to treat, ameliorate, and palliate pre-existing conditions. Where lung function is impaired due to clogging of the airways by secretions, ODSH can be combined with one or more therapeutic agents intended to clear secretions, such as mucolytic agents, including deoxyribonucleases (DNases). Where lung function is impaired due to constriction of the airway, it may be desirable to administer a bronchodilator. Other therapeutic agents that may be combined with ODSH include anti-inflammatory agents, steroids, or beta-agonists.

The specific therapeutic agent(s) selected will depend on the condition to be treated. In the context of treating or preventing an infection, such as a Pseudomonas infection, of the lung in a patient suffering from COPD, ODSH may be administered in combination with one or more therapeutic agents, including bronchodilators, corticosteroids, and oxygen.

In the context of treating or preventing an infection, such as a Pseudomonas infection, of the lung in a patient suffering from cystic fibrosis (CF), ODSH may be combined with a mucolytic agent, such as a DNase, bronchodilating agents, anti-inflammatory agents and/or antibiotics as described above. See, e.g., Gibson et al., 2003, Am. J. Respir. Crit. Care Med. 168:918-951 and Doring et al., 2000, Eur. Respir. J. 16:749-767, describing agents used to treat Pseudomonas infections in CF patients as well as standard regimens of other therapeutic agents used in CF patients, the disclosures of each of which are incorporated herein in their entireties. In a specific example, ODSH is administered in combination with human recombinant DNase I, e.g., dornase alfa. Dornase alfa is marketed under the brand name Pulmozyme®.

ODSH may be administered parenterally, by inhalation, or even by intratracheal injection. Parenteral administration may be subcutaneous or intravenous. In certain embodiments, ODSH is administered intravenously, either as a bolus, as a continuous infusion, or as a bolus followed by continuous infusion. When administered by inhalation, ODSH can be administered in aerosol particles, by nebulization or by dry powder inhalation. When ODSH is administered in combination with one or more other therapeutic agents, ODSH and the other therapeutic agents can be administrated via the same or via different routes.

Adjunctive administration of ODSH, that is administration of ODSH in combination with one or more other therapeutic agent(s), includes administration concurrently with or and administration separately from other therapeutic agent(s). Administration is said to be concurrent if ODSH is administered simultaneously or sequentially with the other therapeutic agent(s). Administration is said be sequential if ODSH is administered on the same day, but not simultaneously with, the other therapeutic agent(s), for example during the same patient visit. Administration is said to separate if ODSH is administered on a different day from the day the subject receives the other therapeutic agent(s) but during an ongoing treatment regimen. When administered separately or sequentially, ODSH can be administered before, after, or both before and after the other therapeutic agent(s).

In some contexts, it is desirable to administer ODSH at times when a subject is not receiving another therapeutic agent to reduce the burden on the subject's system and/or minimize the number of hours spent receiving treatment and/or to simplify the treatment regimen. In such cases, ODSH can be administered for a first period and then not administered in a second period during which the subject is receiving another therapeutic agent. The first and second periods can be of the same or different duration, and may be, each, a day, several days, a week, several weeks, 28 days, a month, or several months.

Therapeutic regimens for adjunctive administration of ODSH with other therapeutic agent(s) can include combinations of concurrent (simultaneous or sequential), and separate administration, for example, simultaneous administration on certain days, and/or separate on other days, and/or sequential on yet other days.

ODSH is administered for a time and in an amount sufficient to provide a therapeutic or prophylactic effect, as will be described in more detail below.

In various embodiments, ODSH is administered over a period of 2 weeks to indefinitely, a period of 2 weeks to 6 months, a period of 3 months to 5 years, a period of 6 months to 1 or 2 years, or the like. Optionally, ODSH administration can be repeated, for example, once daily, twice daily, every two days, three days, five days, one week, two weeks, or one month. The repeated administration can be at the same dose or at a different dose.

Where ODSH is administered in combination with one or more other therapeutic agents, e.g., antibiotics, anti-microbials, mucolytic agents, DNases, bronchodilators, anti-inflammatory agents, steroids, etc., the other therapeutic agent(s) is administered according to standard regimens (dose, route of administration, duration and frequency of treatment, etc.), known to those skilled in the art, for the specific agent being administered.

In some embodiments, ODSH and antibiotic or anti-microbial agents can be administered parenterally. In some embodiments, antibiotic or anti-microbial agents, for example, but not limited to, aztreonam, tobramycin, and fluoroquinolones (e.g., ciprofloxacin), are administered by inhalation. In a specific example, ODSH and tobramycin are administered by inhalation. Patients receive a 300 mg nominal dose tobramycin, administered as an aerosol of a 5 ml dose with a standard jet nebulizer twice daily, on a 28 day “on” therapy followed by a 28 day “off” period, to reduce the potential for development of resistant bacterial strains. See, also, U.S. Pat. No. 5,508,269, the disclosure of which is incorporated herein in its entirety by reference.

In some embodiments, ODSH is administered in combination with a DNase. In a specific embodiment, ODSH is administered via inhalation in combination with a recombinant human DNase, e.g., dornase alfa. Treatment regimens and dosing information for dornase alfa is known in the art. See, e.g., Product Information, Pulmozyme® (dornase alfa) inhalation solution.

For purposes of treating a pulmonary infection, such as a Pseudomonas infection, ODSH is administered to a subject suffering from a Pseudomonas infection in a therapeutically effective (or therapeutic) amount. A therapeutically effective amount is an amount sufficient or effective to provide a therapeutic benefit. A therapeutic benefit can be inferred if one or more of the following is achieved: improvement in any of the symptoms associated with infection of the lung, reduction in the bacterial load in the lung, reduction or elimination of inflammatory or immune, e.g., neutrophil, cells from lung sputum and/or bronchoalveolar lavage fluid, reduction in protein content in the lungs, reduction in HMGB 1 in the lungs, improved survival of the host and enhanced host immunity. A complete cure, while desirable, is not required for therapeutic benefit to exist. In some contexts, a therapeutic benefit can be correlated with one or more surrogate end points, in accordance with the knowledge of one of ordinary skill in the art. By way of example and not limitation, reducing bacterial load in an appropriate animal model is indicative of therapeutic benefit. See, e.g., Example 1.

For purposes of preventing a future pulmonary infection, such as a Pseudomonas infection, a prophylactically effective (or prophylactic) amount of ODSH can be administered to a subject at risk for developing an infection in the lung. In the context of prophylaxis, a prophylactic amount is an amount that would provide a therapeutic benefit if administered to a patient suffering from a pulmonary infection. As used herein, an effective amount of ODSH includes a therapeutically effective (or therapeutic) amount and a prophylactically effective (or prophylactic) amount.

The amount of ODSH administered will depend on various factors, including the severity of the infection being treated, the form, route, and site of administration, the treatment regimen (for example, whether another therapeutic agent is used in addition to ODSH), the age and condition of the subject being treated, including the presence of complicating factors such as other diseases or conditions. The appropriate dosage can be readily determined by a person of skill in the art. In practice, a physician will determine appropriate dosages to be used. This dosage can be repeated as often as appropriate. The amount and/or frequency of the dosage can be altered, increased, or reduced, depending on the subject's response and in accordance with standard clinical practice. The proper dosage and treatment regimen can be established by monitoring the progress of therapy using conventional techniques known to people skilled in the art.

Effective dosages can be estimated initially from in vitro assays or in vivo assays in animals. For example, an initial dose used in animals may be formulated to achieve a desired circulating blood or serum concentration of ODSH when administered parenterally. Calculating dosages to achieve such circulating blood or serum concentrations taking into account bioavailability of ODSH is well within the capabilities of skilled artisans. Similarly, doses can also be formulated to achieve a certain concentration of ODSH that results in an effective dosage in the lung, in particular the deep lung and/or the upper airways, when administered by inhalation. Ordinarily skilled artisans can routinely adapt information derived from relevant animal models useful for testing the efficacy of compounds, to determine dosages suitable for human administration. See, e.g., Example 1 below for an animal model testing efficacy in a mouse model of Pseudomonas-induced lung infection. Further guidance can be found, for example, in Fingl & Woodbury, “General Principles” in Goodman and Gilman's The Pharmaceutical Basis of Therapeutics, Chapter 1, latest edition, Pagamon Press, and references cited therein.

In some embodiments, ODSH is administered at a dose or amount per kilogram of patient body weight ranging from about 1.0 mg/kg to about 50.0 mg/kg, or about 1 mg/kg to about 25 mg/kg for bolus doses, and from about 0.1 mg/kg/hr to about 2.5 mg/kg/hr for infusions, or from about 25 mg to 800 mg in volumes of 2.0 mL or less per injection site, for subcutaneous injections.

In one or more embodiments, ODSH is administered by inhalation and a therapeutic or prophylactic dose is delivered to a subject in need thereof in three or four inhalations or less, such as in two inhalations, or in a single inhalation. In preferred embodiments, a therapeutic or prophylactic dose of ODSH is delivered to a patient in need thereof in three inhalations or less, such as in two inhalations, or in a single inhalation. In some embodiments, a therapeutic or prophylactic dose of ODSH is delivered in less than about three minutes, preferably less than about two minutes, or less than about one minute. In some embodiments, pharmaceutical compositions for inhalation are suitable to provide a dose of ODSH ranging from about 25 mg to about 800 mg per administration.

Other therapeutic agents as described herein can be administered in effective amounts, according to known dosing regimens.

4.2. ODSH and Other Low-Anticoagulant Heparinoids

In certain embodiments, the subject with a pulmonary infection is treated with ODSH or another low-anticoagulant heparinoid. In certain embodiments, a low-anticoagulant heparinoid other than ODSH may be used in any of the methods or compositions described herein.

“Low-anticoagulant heparinoids”, as used herein, are linear glycosaminoglycan polymers made up of alternating or repeating iduronic acid and glucosamine units bearing O-sulfate, N-sulfate, and N-acetyl substitutions. Preferably, low-anticoagulant heparinoids for use in the methods described herein are polymers having an average molecular weight of at least about 8 kDa, for example having an average molecular weight ranging from about 8 kDa to about 15 kDa. In certain embodiments, the low-anticoagulant heparinoids have an average molecular weight of greater than about 8 kDa. More preferably, low-anticoagulant heparinoids for use in the methods described herein have an average molecular weight that ranges in size from about 11 kDa to about 13 kDa.

The low-anticoagulant heparinoids may have an average molecular weight from about 2 kDa to about 15 kDa. In certain embodiments, the low-anticoagulant heparinoids have an average molecular weight of at least about 2 kDa, at least about 3 kDa, at least about 4 kDa, at least about 5 kDa, at least about 6 kDa, or at least about 7 kDa. In certain embodiments, the low-anticoagulant heparinoids have an average molecular weight of less than about 15 kDa, less than about 14 kDa, less than about 13 kDa, less than about 12 kDa, less than about 11 kDa, less than about 10 kDa, or less than about 9 kDa. In some embodiments, the average molecular weight of the low-anticoagulant heparinoid is selected from about 2 kDa, 3 kDa, 4 kDa, 5 kDa, 6 kDa, 7 kDa, 8 kDa, 9 kDa, 10 kDa, 11 kDa, 12 kDa, 13 kDa, 14 kDa, 15 kDa, 16 kDa, 17 kDa, 18 kDa or a range including any of these values as endpoints. Molecular weight of heparinoids can be determined by high performance size exclusion chromatography as is known in the art. See, e.g., Lapierre et al., 1996, Glycobiology 6(3):355-366, at page 363; Fryer et al., 1997, J. Pharmacol. Exp. Ther. 282: 208-219, at page 209.

The low-anticoagulant heparinoids used in the methods described herein have reduced anticoagulant activity or are substantially non-anticoagulant. Low anticoagulant heparinoids have no more than 40% of the anti-coagulant activity of an equal weight of unfractionated heparin. For example, the low-anticoagulant heparinoid has no more than 35%, no more than 30%, no more than 20%, even no more than 10%, 9%, 8%, 7%, 6%, 5%, 4%, 3%, 2% or 1% of the anti-coagulant activity of an equal weight of unfractionated heparin. In certain embodiments, the low-anticoagulant heparinoids interact with Platelet Factor 4 (PF4), for example, the heparinoids bind to PF4.

Anticoagulant activity can be determined using assays known in the art. In certain embodiments, anticoagulant activity is determined by activated partial thromboplastin time (aPTT) assay. In some embodiments, anticoagulant activity is determined by assay of prothrombin time. In particular embodiments, anticoagulant activity is determined by anti-Xₐ activity. In a variety of embodiments, anticoagulant activity is determined by clotting assay. In some embodiments, anticoagulant activity is determined by amidolytic assays. In certain embodiments, anticoagulant activity is determined by the USP assay. See, e.g., U.S. Pat. No. 5,668,118, Example IV; Fryer et al., 1997, J. Pharmacol. Exp. Ther. 282: 208-219, at page 209; Rao et al., 2010, Am. J. Physiol. 299:C97-C110, at page C98; United States Pharmacopeia Convention 1995 (for USP anti-coagulant assay and amidolytic assay).

A low-anticoagulant heparinoid used in the methods described herein is low-anticoagulant in at least one of the above-described assays. In certain embodiments, the low-anticoagulant heparinoid used in the methods described herein is low-anticoagulant in more than one of the above-described assays.

In a variety of embodiments, the low-anticoagulant heparinoid is one which exhibits substantially reduced anti-Xₐ activity, which can be determined in an assay carried out using plasma treated with Russell viper venom.

In specific embodiments, the low-anticoagulant heparinoid used in the methods described herein is ODSH. ODSH has been demonstrated to exhibit less than 9 U of anti-coagulant activity/mg in the USP anti-coagulant assay (e.g., 7±0.3 U), less than 5 U of anti-Xₐ activity/mg (e.g., 1.9±0.1 U/mg) and less than 2 U of anti-IIₐ activity/mg (e.g., 1.2±0.1 U/mg). Unfractionated heparin has an activity of 165-190 U/mg in all three assays. See Rao et al., 2010, Am. J. Physiol. 299:C97-C110, page C101. In addition, ODSH has a low affinity for anti-thrombin III (Kd˜339 μM or 4 mg/ml vs. 1.56 μM or 22 μg/ml for unfractionated heparin), consistent with the observed low level of anti-coagulant activity, measured as described in Rao et al., supra, at page C98.

In typical embodiments, the low-anticoagulant heparinoids are partially desulfated. Preferably, the low-anticoagulant heparinoids are substantially desulfated at the 2-O position of α-L-iduronic acid (referred to herein as the “2-O position”) and/or desulfated at the 3-O position of D-glucosamine-N-sulfate (6-sulfate) (referred to herein as the “3-O position”). In some embodiments, the low-anticoagulant heparinoids are at least 85%, at least 90%, at least 95%, at least 96%, at least 97%, at least 98%, or at least 99% desulfated at the 2-O position. In selected embodiments, the low-anticoagulant heparinoids are at least 99% desulfated at the 2-O position. In some embodiments, the low-anticoagulant heparinoids are at least 85%, at least 90%, at least 95%, at least 96%, at least 97%, at least 98%, or at least 99% desulfated at the 3-O position. In selected embodiments, the low-anticoagulant heparinoids are at least 99% desulfated at the 3-O position. In some embodiments, the low-anticoagulant heparinoids are at least 85%, at least 90%, at least 95%, at least 96%, at least 97%, at least 98%, or at least 99% desulfated at both the 2-O position and the 3-O position. In selected embodiments, the low-anticoagulant heparinoids are at least 99% desulfated at the 2-O position and the 3-O position.

In typical embodiments, the low-anticoagulant heparinoid comprises substantially N-sulfated and 6-O sulfated D-glucosamine. In some embodiments, the carboxylates on α-L-iduronic acid sugars of low-anticoagulant heparinoid are substantially intact.

An exemplary low-anticoagulant heparinoid is substantially 2-O, 3-O desulfated heparin, referred to herein as ODSH. ODSH for use in the above-described methods can be prepared from bovine or porcine heparin. In an exemplary method of preparing ODSH from porcine heparin, ODSH is synthesized by cold alkaline hydrolysis of USP porcine intestinal heparin, which removes the 2-O and 3-O sulfates, leaving N- and 6-O sulfates on D-glucosamine sugars and carboxylates on α-L-iduronic acid sugars substantially intact. Fryer, A. et al., 1997, J. Pharmacol. Exp. Ther. 282: 208-219. Using this method, ODSH can be produced with an average molecular weight of about 11.7±0.3 kDa, and low affinity for anti-thrombin III (Kd=339 μM or 4 mg/ml vs. 1.56 μM or 22 μg/ml for heparin), consistent with the observed low level of anticoagulant activity.

Methods for the preparation of 2-O, 3-O desulfated heparin may also be found, for example, in U.S. Pat. Nos. 5,668,118, 5,912,237, and 6,489,311, and WO 2009/015183, the contents of which are incorporated herein in their entirety, and in U.S. Pat. Nos. 5,296,471, 5,969,100, and 5,808,021.

4.3. Pharmaceutical Compositions and Unit Dosage Forms

Also provided herein are pharmaceutical compositions and unit dosage forms for use in the methods of treating and methods of preventing pulmonary infection, such as a Pseudomonas infection. The pharmaceutical compositions comprise an effective amount of ODSH, optionally in combination with one or more additional therapeutic agents as described above, and are in a form suitable for the desired mode of administration. Pharmaceutical compositions may further comprise one or more pharmaceutically acceptable buffers, diluents, excipients, carriers, preservatives, and/or other non-therapeutic components, as will be described further below. The specific components used will depend on the desired mode of administration.

Depending on the intended mode of administration, the pharmaceutical compositions may be in the form of solid, semi-solid, or liquid dosage forms, such as, for example, a dry powder or a liquid for aerosol inhalation. Preferably, the pharmaceutical compositions may be sterile. In some embodiments, the pharmaceutical compositions can be in the form of a sterile, non-pyrogenic, fluid composition.

For parenteral administration, compositions will typically be formulated as an injectable. Injectables can be prepared in conventional forms, either as liquid solutions or suspensions, solid forms suitable for solution or suspension in liquid prior to injection, or as emulsions. Pharmaceutical formulations for parenteral administration can be suitable for subcutaneous or intravenous injection. Another approach for parenteral administration involves use of a slow release or sustained release system, such that a constant level of dosage is maintained. See, e.g., U.S. Pat. No. 3,710,795, which is incorporated by reference herein in its entirety.

Pharmaceutical compositions can also be formulated for administration by inhalation. Such formulations can comprise aerosol particles of less than 10 microns, preferably less than 5 microns, more preferable about 1 to about 5 microns. Such aerosol particles can be in an aqueous formulation suitable for delivery by available jet aerosol or ultrasonic nebulizer systems in common use or in a powder formulation suitable for delivery by dry powder inhalation systems known in the art.

For applications where ODSH is administered by inhalation, it may be desirable to administer ODSH and a second, and optionally third, fourth, fifth, etc., therapeutic agent(s) using the same inhalation device. For such applications, ODSH can be formulated as a separate composition or in the same composition as the second, and optionally third, fourth, fifth, etc., therapeutic agent(s).

In some embodiments, ODSH is formulated in a pharmaceutical composition with sodium chloride, and, optionally, other chloride salts. For example, ODSH can be formulated in a pharmaceutical composition comprising between 0.1 and 2.5 mg/ml sodium chloride. In some instances, the pharmaceutical composition will contain 0.15 mg/ml calcium chloride dehydrate and 8.77 mg/ml sodium chloride.

In a specific exemplary embodiment, ODSH is formulated in a pharmaceutical composition that includes 1.0 mg/ml dornase alfa. Preferably, the pharmaceutical composition also includes 0.15 mg/ml calcium chloride dehydrate and 8.77 mg/ml sodium chloride.

In another specific exemplary embodiment, ODSH is formulated in a pharmaceutical composition that includes 60 mg/mL tobramycin solution for inhalation. In some embodiments, ODSH is formulated in a pharmaceutical composition including 2.25 mg/ml sodium chloride.

In other specific embodiments, ODSH is formulated in a pharmaceutical composition suitable for inhalation that includes ciprofloxacin, aztreonam, or levofloxacin.

ODSH can be administered, e.g., as a complex with cationic liposomes, or encapsulated in anionic liposomes.

Liquid pharmaceutically administrable compositions can, for example, be prepared by dissolving, dispersing, etc. an active compound as described herein and optional pharmaceutical adjuvants in an excipient, such as, for example, water, saline, aqueous dextrose, glycerol, ethanol, and the like, to thereby form a solution or suspension. If desired, the pharmaceutical composition to be administered may also contain minor amounts of nontoxic auxiliary substances such as wetting or emulsifying agents, pH buffering agents and the like, for example, sodium acetate, sorbitan monolaurate, triethanolamine sodium acetate, triethanolamine oleate, etc.

Pharmaceutical compositions of ODSH can be formulated in an amount that permits bolus intravenous administration and/or continuous intravenous infusion at appropriate doses, as described above. In one embodiment, the pharmaceutical composition comprises ODSH at a concentration of 50 mg/mL. When formulated for subcutaneous administration, pharmaceutical compositions can contain ODSH at a concentration ranging from 50 mg/ml to 800 mg/ml suitable for administration at doses ranging from about 25 to about 800 mg, in volumes of 2.0 mL or less per injection site.

Liquid compositions can be aerosolized for administration. Actual methods of preparing such dosage forms are known, or will be apparent, to those skilled in this art; for example, see Remington's Pharmaceutical Sciences, E. W. Martin, (ed.), Mack Publishing Co., Easton, Pa.

In some embodiments, the pharmaceutical composition is in powder form and is administered using a dry powder inhaler. See, e.g., U.S. Published Application Nos. 20020017295 and 20040105820, PCT publication WO 02/83220, and U.S. Pat. No. 6,546,929. Alternatively, the pharmaceutical composition is administered using a nebulizer, as described in PCT publication WO 99/16420.

Aerosolization of the pharmaceutical formulation may be accomplished by any means known in the art, including by pressurized gas flowing through the inlets, as described for example in U.S. Pat. No. 5,458,135, U.S. Pat. No. 5,785,049, and U.S. Pat. No. 6,257,233, or propellant, as described in PCT publication WO 00/72904 and U.S. Pat. No. 4,114,615. All of the above references being incorporated herein by reference in their entireties.

Pharmaceutical compositions can be conveniently presented in unit dosage forms. Unit dosage forms contain ODSH in amounts and volumes suitable for administration in the effective doses as described herein.

Unit dosage forms can contain for example, but without limitation, 1 mg to 1 g, or 5 mg to 500 mg of ODSH. In some embodiments, unit dosage forms contain a single dose. In some embodiments, unit dosage forms contain multiple doses. Typically, unit dosage forms can range in volume from 1 mL to 1000 mL, for example, 0.1 mL, 0.2 mL, 0.3 mL, 0.5 mL, 1 mL, 1.5 mL, 2 mL, 2.5 mL, 3 mL, 3.5 mL, 4 mL, 4.5 mL, 5 mL, 10 mL, 30 mL, 100 mL, 200 mL, 500 mL, or 1000 mL, or any intermediate volume.

In addition to ODSH, unit dosage forms can also include one or more additional therapeutic agents in amounts and volumes suitable for administration in effective doses. In some embodiments, a unit dosage form comprises ODSH and a human DNase, e.g., dornase alfa. In such embodiments, the unit dosage form can be a single-use ampule suitable for use in a suitable aerosolization device, e.g., a nebulizer. In some embodiments, ODSH and a human DNase, e.g., dornase alfa can be in separate unit dosage forms, where the unit dosage forms are suitable for use in the same aerosolization device.

In some embodiments, a unit dosage form comprises ODSH and an antibiotic, e.g. tobramycin, ciprofloxacin, or aztreonam. In such embodiments, the unit dosage form can be a single-use ampule suitable for use in a suitable aerosolization device, e.g., a nebulizer. In some embodiments, ODSH and the antibiotic can be in separate unit dosage forms, where the separate unit dosage forms are suitable for use in the same aerosolization device.

Unit dosage forms have containers appropriate for the volumes and intended route of administration. Unit dosage forms suitable for parenteral administration include, for example, ampules, vials, preloaded syringes, infusion bags, cartridge units with Luer lock connectors, single or multidose containers.

Unit doses may be provided within containers, e.g., a capsule or an ampule, that can be inserted into the aerosolization device. Such containers will be of suitable shape, size, and material to contain the pharmaceutical composition and provide the pharmaceutical formulation in a useable condition. For example, the container may comprise a wall which comprises a material that does not adversely react with the pharmaceutical formulation. In addition, the wall may comprise a material that allows the capsule to be opened to allow the pharmaceutical formulation to be aerosolized. In one version, the wall comprises one or more of gelatin, hydroxypropyl methylcellulose (HPMC), polyethyleneglycol-compounded HPMC; hydroxyproplycellulose, agar, or the like. In one version, the container may comprise telescopically adjoining sections, as described for example in U.S. Pat. No. 4,247,066 which is incorporated herein by reference in its entirety.

The size of the container may be selected to adequately hold the dose of the pharmaceutical formulation. The container can have an outer diameter ranging from about 4.91 mm to 9.97 mm, a height ranging from about 11.10 mm to about 26.14 mm, and a volume ranging from about 0.13 ml to about 1.37 ml, respectively. Suitable containers are available commercially from, for example, Shionogi Qualicaps Co. in Nara, Japan and Capsugel in Greenwood, South Carolina. After filling, a top portion may be placed over the bottom portion to form a capsule shape and to contain the pharmaceutical composition within the container, as described in U.S. Pat. No. 4,846,876, U.S. Pat. No. 6,357,490, and in PCT publication WO 00/07572, all of which are incorporated herein by reference in their entireties.

Unit dosage forms for inhalation can be administered by way of an aerosolization device, which can be a dry powder inhaler or a nebulizer. Any suitable dry powder aerosolization device may be used, including, but not limited to, those described in U.S. Pat. No. 4,069,819, U.S. Pat. No. 4,995,385, U.S. Pat. No. 3,991,761, U.S. Pat. No. 4,338,931, U.S. Pat. No. 5,619,955, U.S. Pat. No. 7,559,325, U.S. Pat. No. 7,516,741, U.S. Published Application Nos. 20030150454, 20030094173, 20050000518, 20040206350, and 20030106827, all of which are incorporated herein by reference in their entireties. Suitable nebulizers are also known in the art and commercially available, such as, for example, Hudson T Up-draft II®, Marquest Acorn II®, PART LC Jet+, PART BABY, or Durable Sidestream®.

Unit dosage forms may be suitable or adapted for use in any commercially available aerosolization device, including devices sold or marketed under the following tradenames and/or trademarks: Handihaler (Boehringer Ingelheim), Eclipse (Aventis), AIR inhaler (Alkermes), Cyclohaler (Plastiape), Concept 1 (Novartis), Flowcaps (Hovione), Turbospin (PH&T), Monohaler (Pfizer), Spinhaler (Aventis), Rotahaler (GSK). Suitable blister-based inhalers include: the Diskus and Gemini (GSK), the device of Nektar Therapeutics disclosed in PCT publication WO02/022830, which is incorporated herein by reference, Gyrohaler (Vectura), E-Flex, Microdrug, Diskhaler (GSK). Other suitable active dry powder inhalers include: the Exubera® inhalation device, which is described in U.S. Pat. No. 6,257,233, incorporated herein by reference, Aspirair (Vectura), and Microdose inhaler (Microdose).

4.4. Kits

The pharmaceutical and unit dosage forms described herein may conveniently be provided in a kit. A kit can contain one or more individually packaged unit doses of ODSH and instructions for use. In some embodiments, the kit will also contain one or more additional therapeutic agents, e.g., a second, a third, a fourth, etc., therapeutic agent, formulated in unit doses suitable for administration in combination with ODSH. In kits that provide ODSH and one or more additional therapeutic agents, the ODSH and additional therapeutic agent(s) can be formulated for administration by the same route or by a different route. In embodiments where the ODSH and one or more additional therapeutic agents, the ODSH and additional therapeutic agent(s) are formulated for administration by the same route, the therapeutic agents can further be formulated for administration using the same device.

In some embodiments, kits will include ODSH and a second, a third, a fourth, etc., therapeutic agent formulated for administration by inhalation. In some instances, ODSH and the additional therapeutic agent(s) are provided in separate unit dosage forms. In some instances, ODSH and the additional therapeutic agent(s) are formulated in the same unit dosage form, and a kit may contain one or more unit dosage forms. In an specific embodiment, a kit contain one or more unit dosage form of ODSH and one or more unit dosage form of a second therapeutic agent, e.g., dornase alfa, formulated for administration using the same inhalation device.

### 5. EXAMPLES

### Example 1

### ODSH Reduces Pseudomonas aeruginosa Load in Lungs of Mice Infected with the Pathogen

This experiment demonstrates the effect of ODSH in reducing bacterial load in mice inoculated with Pseudomonas aeruginosa.

1.1 Materials & Methods

Male C57BL/6 mice (8-12 weeks old) were inoculated with 5×10⁸ CFU P. aeruginosa (non-mucoid Green Fluorescent Protein-labeled P. aeruginosa strain PAO1) via oropharyngeal aspiration, causing severe pulmonary infection and substantial lung injury with marked neutrophil recruitment 24 h following the inoculation.

Mice were treated with 16.4, 49.3, or 148 mg/kg O-desulfated heparin (ODSH), administered subcutaneously at 5 minutes before and 12 hours after inoculation with bacteria. 17 to 23 mice were included in each treatment group (including a control group receiving no ODSH).

24 hours after infection, mice were euthanized, their lungs were isolated. Viable bacteria in the lungs were quantified by plating serial dilutions of homogenized lungs and expressed as log scale of the mean (+/−SEM) of colony-forming units (CFU) per lung. Results are based on seven independent experiments with 15 to 21 mice per group. Statistical significance (p<0.05) was determined relative to the control group.

1.2 Results

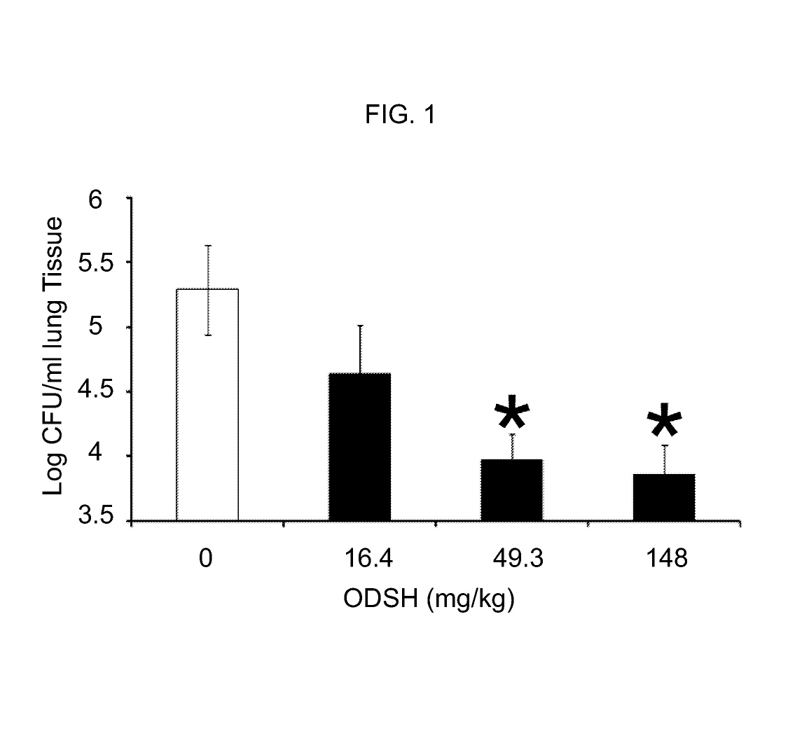

As shown in FIG. 1, bacterial burden in lungs of mice that received ODSH at 49.3 and 148 mg/ml was markedly reduced. There is approximately a 50 fold reduction in the bacterial load in the lung in the treated mice compared to that of the control mice. Thus, ODSH can effectively enhance the host defense to clear bacterial infection in bacterial pneumonia. In addition, mice received ODSH, at 49.3 mg/ml, were observed to be active and healthy, as compared to mice in the control group and those treated with ODSH at 16.4 mg/ml, which exhibited clinical signs of illness, including lethargy and huddling together in the corners of their cages.

### Example 2

### ODSH Reduces Pulmonary Bacterial Burden in Mice with Pseudomonas aeruginosa (P. aeruginosa)

This experiment further demonstrates the effect of ODSH in reducing bacterial burden in mice inoculated with P. aeruginosa.

2.1 Materials & Methods

Male C57BL/6J mice (8-12 weeks old, The Jackson Labs, Bar Harbor, Me.) were inoculated via intranasal administration with 5×10⁸ CFU/mouse of PAO1, a non-mucoid strain of P. aeruginosa. At 0 and 12 hr post inoculation the infected mice were administered 25 or 75 mg/kg ODSH or saline by subcutaneous injection.

Mice were euthanized at fixed timepoints post-infection by intraperitoneal injection of an overdose of sodium pentobarbital (120 mg/kg) to permit harvest of bronchoalveolar lavage (BAL) fluid and lung tissues.

BAL samples were prepared as described in Entezari et al., 2012, Mol. Med., 18:477-485 and Patel et al., 2013, Am. J. Respir. Cell Mol. Biol. 48:280-287. After mice were euthanized, the trachea was exposed and dissected, and a 20-Gauge (×1.25 in) catheter was inserted. The lungs were then gently lavaged twice with two 1 mL volumes of sterile non-pyrogenic phosphate-buffered saline solution (PBS, pH 7.4) (Mediatech, Inc., Herndon, Va.). The BAL samples from each given mouse were combined and centrifuged (1600 rpm, 10 min, 4° C.). The resultant supernatants were collected and stored in a −80° C. freezer for later analyses of levels of HMGB1 and total protein content via Western blot analysis and a bicinchonic acid assay (BCA), respectively. After the lavage, the lungs were harvested and homogenized in 1 mL PBS or fixed with 4% formaldehyde solution and stored in formaldehyde and subsequently stained with hematoxylin and eosin.

Viable bacterial counts in the airways and lungs were determined as described in Entezari et al., 2012, Mol. Med., 18:477-485 and Patel et al., 2013, Am. J. Respir. Cell Mol. Biol. 48:280-287. Viable bacterial counts in the airways and lungs were determined by plating serial dilutions of the BAL and lung homogenates, respectively, onto Pseudomonas Isolation Agar (PIA, Difco; Sparks, Md.) and culturing at 37° C. Each dilution was plated in duplicates. After approximately 16 hours, the numbers of colonies on each plate were enumerated and total colony forming units her milliliter homogenate or BAL were calculated.

2.2 Results

Results are presented as means (±SEM) from at least three independent experiments. The data were analyzed for statistical significance according to paired and unpaired t-tests, analysis of variance (ANOVA), or Kaplan-Meier analysis, using Microsoft Excel. A p-value≦0.05 was considered statistically significant.

The number of viable bacteria in lung homogenates and BAL were assessed to determine bacterial burden in the lungs and airways.

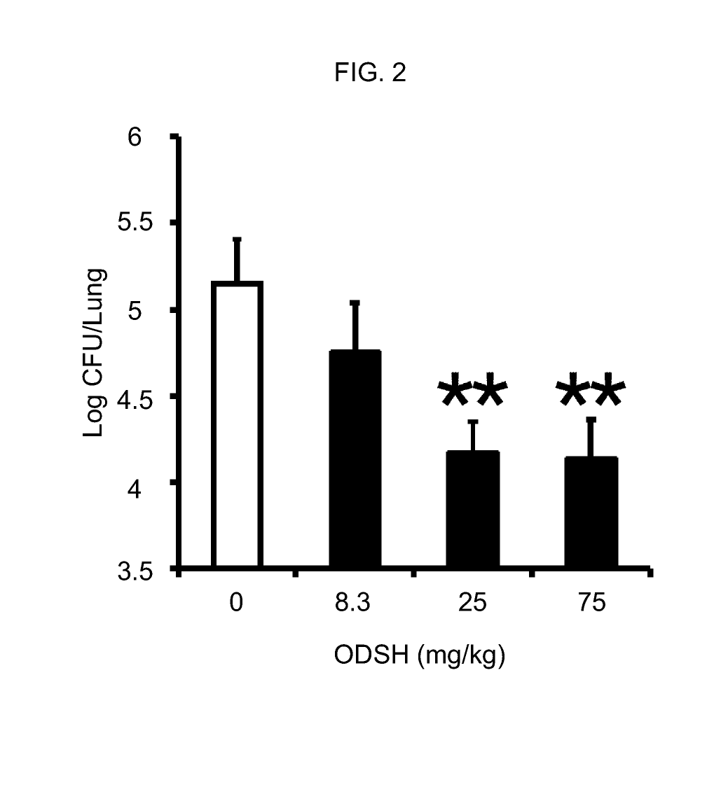

Mice treated with ODSH at 25 and 75 mg/kg showed a significant decrease in bacterial burden in lungs (5.14 [±0.26] vs. 4.16 [±0.18] and 4.13 [±0.22] log CFU/mL lung homogenate, respectively [p≦0.01]) as shown in FIG. 2.

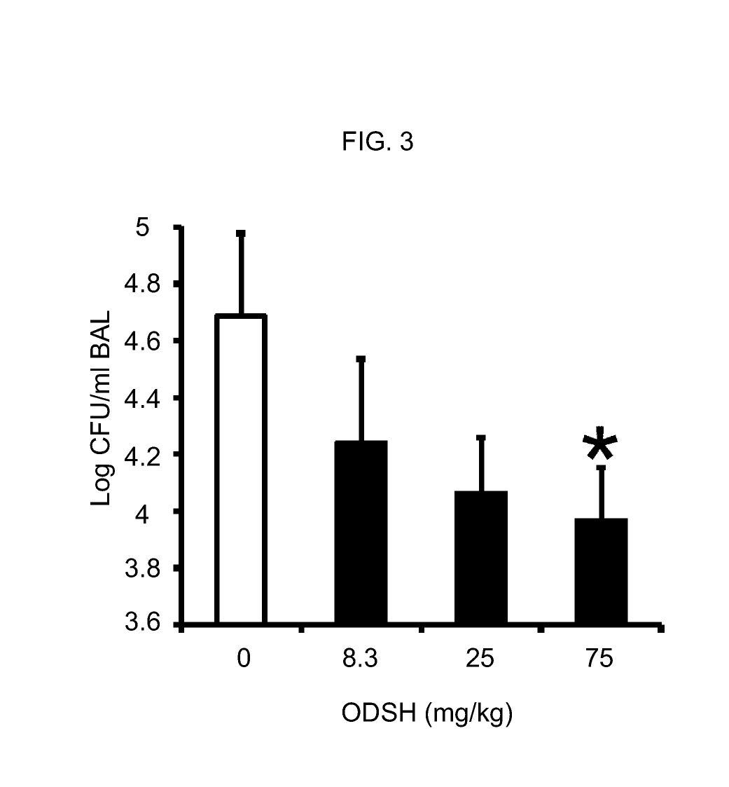

As shown in FIG. 3, the bacterial burden in BAL was similarly reduced upon ODSH treatment (4.68 [±0.29] vs. 4.06 [±0.19] and 3.96 [±0.18] log CFU/mL) at 25 and 75 mg/kg respectively [p≦0.05].

### Example 3

### ODSH Reduces Protein Content, an Indicator of Acute Lung Injury, in the Lungs of Mice Infected with P. aeruginosa

This experiment demonstrates that ODSH reduces acute lung injury in mice inoculated with P. aeruginosa.

3.1 Materials & Methods

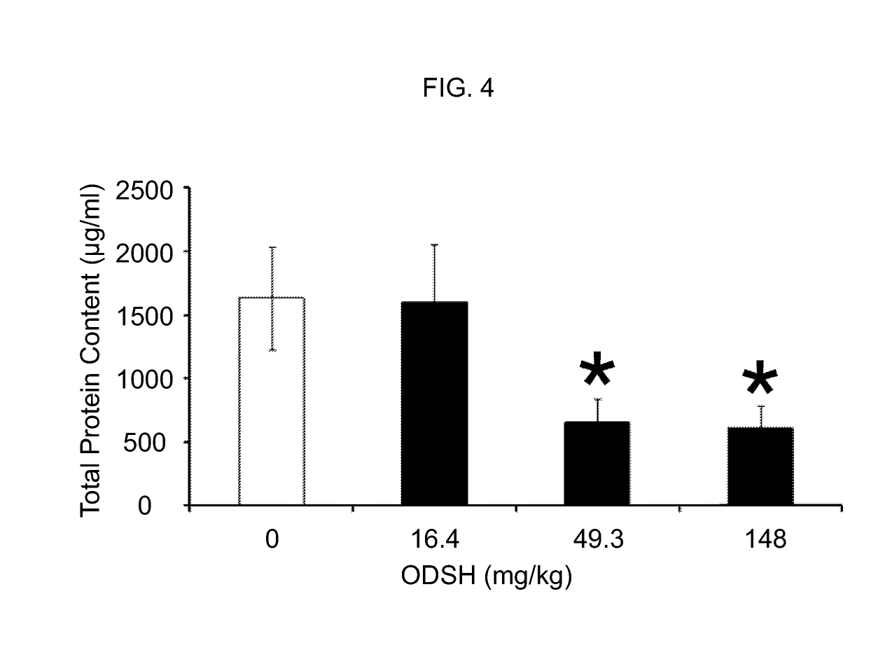

Mice were inoculated with P. aeruginosa and treated with ODSH as described in Example 1 above. After euthanasia and collection of lungs, lung lavage fluid was harvested and total protein content—a marker for lung injury—was determined Values shown in FIG. 4 represent the mean+/−standard error from seven independent experiments, involving 17 to 23 mice per group. Statistical significance (p<0.05) was determined relative to the control group.

3.2 Results

As shown in FIG. 4, ODSH administered at 49.3 and 148 mg/kg significantly reduced the extent of P. aeruginosa-induced lung injury, measured by total protein content in bronchoalveolar lavage fluids. Although 148 mg/kg ODSH significantly reduced the extent of lung injury in mice it also appears to make the mice sick and extensive hemorrhage was observed in these mice.

### Example 4

### ODSH Reduces Protein Content, Ameliorating P. aeruginosa

This experiment further demonstrates that ODSH reduces acute lung injury in mice inoculated with P. aeruginosa.

4.1 Materials & Methods

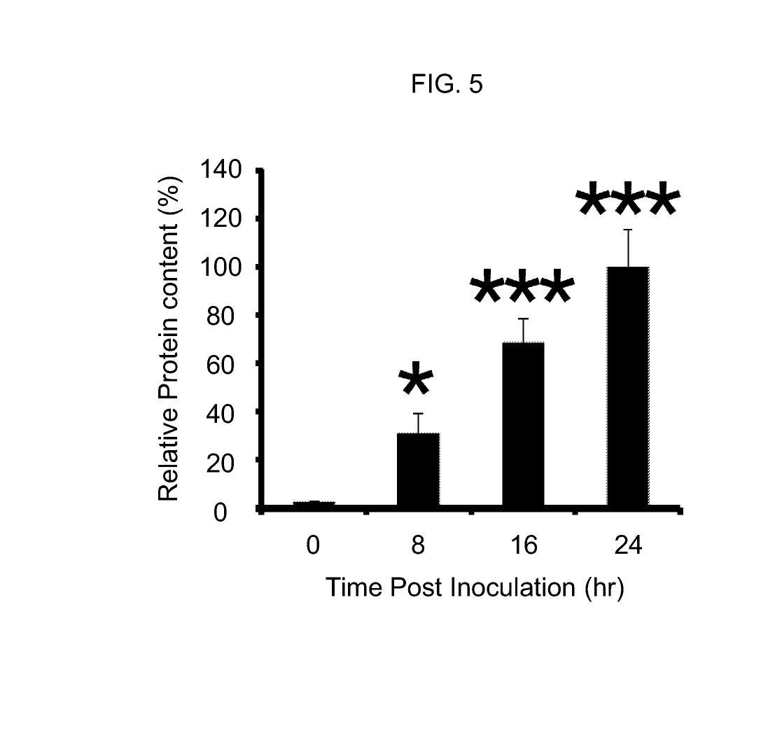

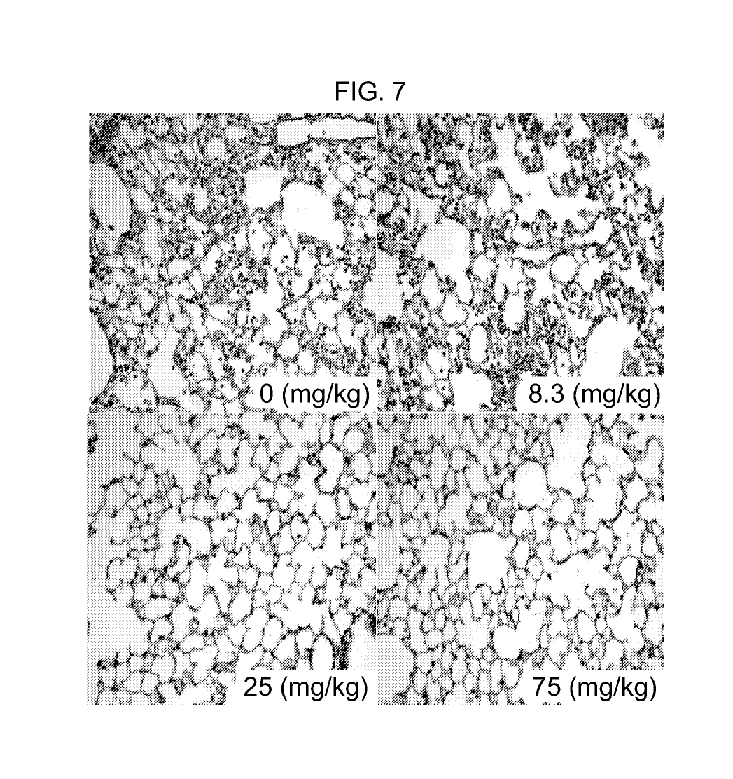

Mice were inoculated with P. aeruginosa and treated with ODSH as described in Example 2 above. After euthanasia and collection of lungs, lung lavage fluid was harvested and total protein content—a marker for lung injury—was determined. As seen in FIG. 5, control mice inoculated with P. aeruginosa (not treated with ODSH) had acute lung injury as indicated by a time-dependent increase of total protein content in airways and marked lung damage at 24 hr post-inoculation (FIG. 7).

BAL total protein concentrations were determined using a colorimetric BCA assay. Total cells in each BAL sample were collected by centrifugation. After the sample's supernatant was removed, the cell pellet was re-suspended into 300 μL of PBS and total cell counts determined using standard hemocytometer procedures. For differential cell counts, cytospin preparations were made and cells were stained with HEMA-3 stain (Fisher Scientific, Kalamazoo, Mich.). Neutrophils were identified by their size and polymorphonuclear nucleus.

4.2 Results

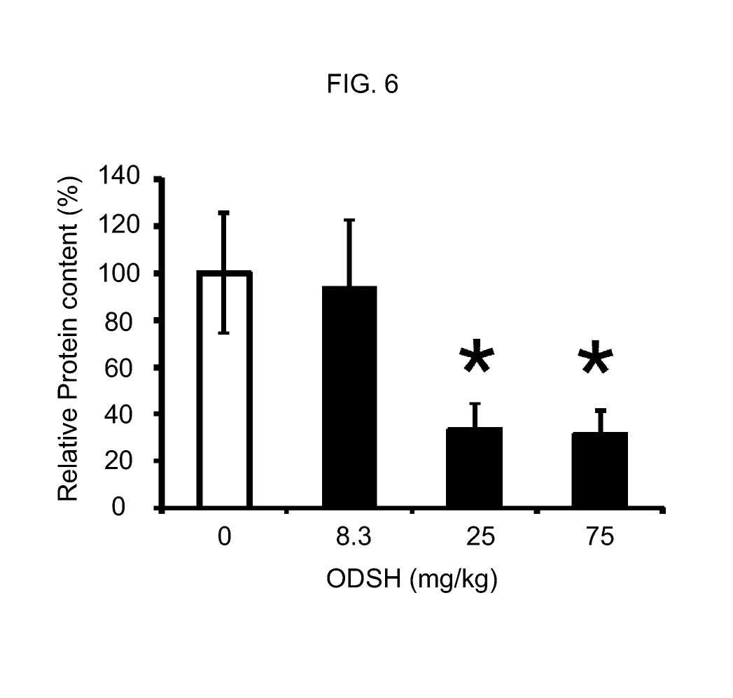

Compared to mice treated with saline, levels of total protein in BAL from ODSH-treated mice were significantly reduced from 100 [±25.52]% of control levels to 33.36 [±10.92]% in mice that received 25 mg/kg ODSH and to 31.22 [±10.02]% in those mice given 75 mg/kg ODSH (p<0.05, FIG. 6). No significant improvement was observed in mice treated with 8.3 mg/kg ODSH (93.34 [±29.05]%).

Histological analysis showed that ODSH at 25 and 75 mg/kg markedly reduced P. aeruginosa-induced cell accumulation in the lung interstitium and alveolar spaces compared to the control animals (FIG. 7). In accordance with histologic images, the number of total cells as well as number of neutrophils was found to be decreased (Table 1) as 24 hr post-intranasal inoculation in ODSH in ODSH treated mice. These data indicate that ODSH administration at 25 and 75 mg/kg helped to maintain alveolar integrity by reducing P. aeruginosa infection-induced lung injury and cell accumulation. In addition, mice treated with ODSH at these levels had improved clinical symptoms compared to the control mice, which exhibited symptoms of severe illness with lethargy and huddling into corners of cages.

TABLE 1
ODSH treatment reduces cell accumulation in the airways

| | ODSH (mg/kg) | | | |
|---|---|---|---|---|
| | 0 | 8.3 | 25 | 75 |
| Total cell count | 20.67 ± 7.35 | 17.04 ± 5.29 | 5.50 ± 2.20 | 5.14 ± 3.48 |
| (×10⁶) | | (p = 0.06) | (p = 0.06) | (p = 0.08) |
| Neutrophil | 5.63 ± 1.74 | 3.15 ± 1.02 | 2.29 ± 0.95 | 1.77 ± 0.74 |
| count (×10⁶) | | (p = 0.38) | (p = 0.11) | (p = 0.06) |

### Example 5

### ODSH Reduces Infiltration of Inflammatory Cells into Lung Fluid in the Lungs of Mice Infected with P. aeruginosa

This experiment demonstrates that ODSH reduces inflammatory responses in the lungs of mice inoculated with P. aeruginosa.

5.1 Materials & Methods

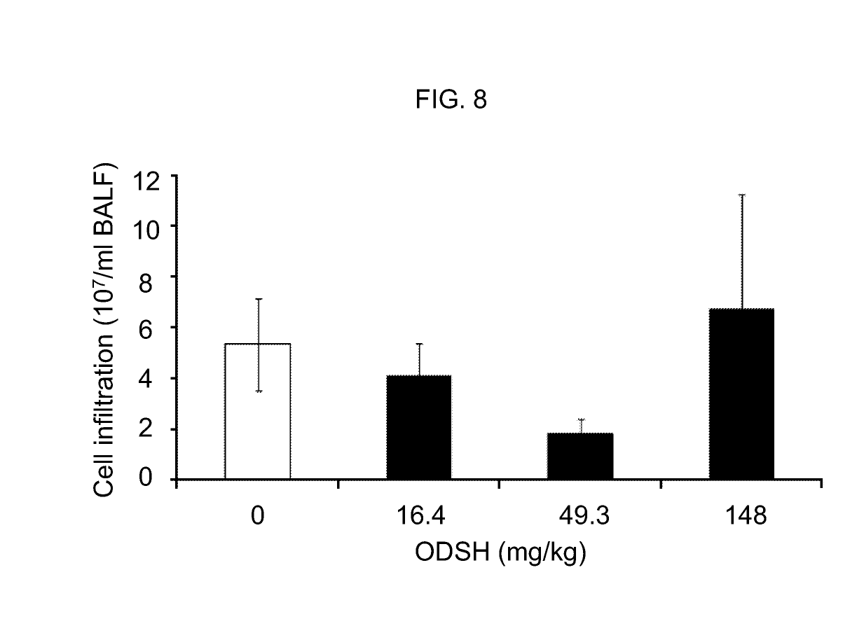

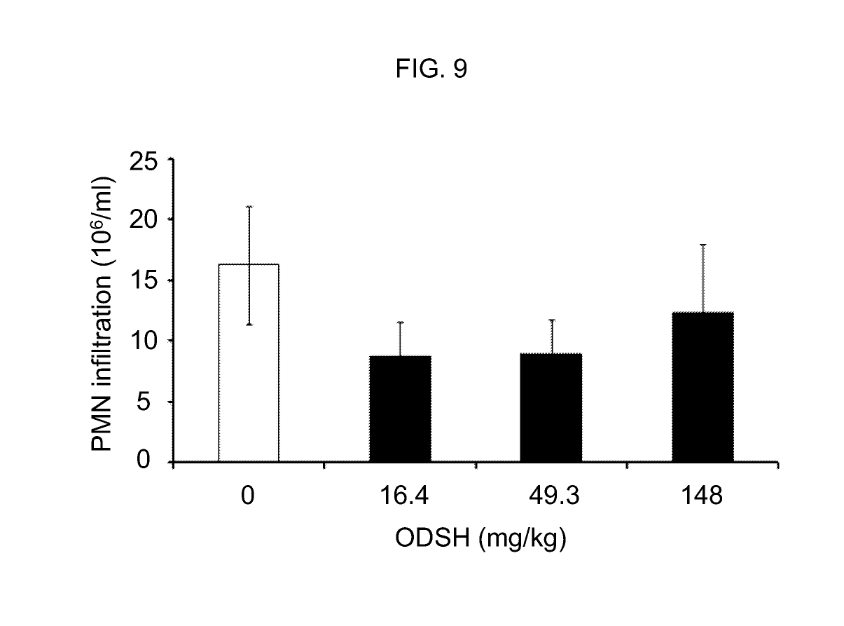

Mice were inoculated with P. aeruginosa and treated with ODSH as described in Example 1 above. Lung lavage fluid was collected from euthanized mice and the number of infiltrated cells, including neutrophils, was determined Data reported in FIGS. 8 and 9 are the mean (+/−standard error) of seven independent experiments. 17 to 23 mice were treated per group.

5.2 Results

ODSH did not significantly affect bacterial infection-induced cell infiltration into the lung at 16.4 mg/kg, but did when administered at 49.3 mg/kg. The number of cell infiltrates in mice treated with 148 mg/kg ODSH was more than that in the control mice (FIG. 8). Similar results were obtained for the number of infiltrated neutrophils (FIG. 9).

### Example 6

### ODSH Reduces P. aeruginosa Infection-Induced Elevation of Airway HMGB1 and Decreases Binding of HMGB1 to TLR2 and TLR4

This experiment demonstrates that ODSH reduces P. aeruginosa infection-induced elevation of airway HMGB1 and decreases binging of HMGB1 to TLR2 and TLR4.

6.1 Materials & Methods

HMGB1 levels from BAL were assessed via immunoblot analysis using anti-HMGB1 antibodies as in Patel et al., 2013, Am. J. Respir. Cell Mol. Biol. 48:280-287. Samples were separated on SDS-PAGE. Proteins were electrotransferred to a PVDF membrane and then blocked in 5% non-fat dry milk in Tris-buffered saline (pH 7.6). The membrane was then incubated with anti-HMGB 1 antibody (1:1000 dilution, Sigma) and then with anti-rabbit horseradish peroxidase-coupled secondary antibodies (1:5000 dilution, GE Healthcare, Princeton, N.J.). After washing, antibody binding was detected using enhanced chemiluminescence plus Western blotting detecting reagents (thermo Scientific, West Palm Beach, Fla.). The blots were then scanned with a UVP Biospectrum 600 Imaging System (vision Works LS, Upland, Calif.) and band intensities quantified using Vision Works image acquisition and analysis software (Version 6.8).

For studies of the effect of ODSH on HMGB1 binding to TLRs, polyvinyl 96-well plates were coated with each TLR of interest (0.5 μg/well). Separately, a constant amount of HMGB1 (0.1 μg HMGB1 protein in 100 μL PBS containing 0.01% Triton X-100 [PBST] and 0.1% BSA) was incubated with and equal volume of serially diluted ODSH (8.5×10⁻⁵-8.5 μM is PBST-0.1% BSA) overnight at 4° C. The following day, 50 μL of HMGB1-ODSH mix was transferred to each TLR-coated well and incubated at 37° C. for 2 hr. Wells were then washed four times with PBST.

To detect bound HMGB1, 50 μL of monoclonal HMGB1-Ab (25 ng/well) was added to each well, the mixture was incubated 1 hr at room temperature, and the wells were washed again four times with PBST. Horseradish peroxidase-conjugated secondary antibody (50 μL/well, 1:2000 dilution) was then added to each well and the plates incubated 1 hr at room temperature. After the wells were washed once with PBST, a colorimetric reaction was initiated by addition of 50 μL TIB solution and terminated after 15 min by addition of 50 μL 1 N HCl. Absorbance at 450 nm was then measured using a Spectromax M2 microplate reader (Molecular Devices, Sunnyvale, Calif.) and plotted against ODSH concentration. The data was analyzed using SoftMax Pro (Molecular Devices) software by fitting the data in a 4-parameter logistic non-linear regression equation to obtain the ICₛₒ values.

6.2 Results

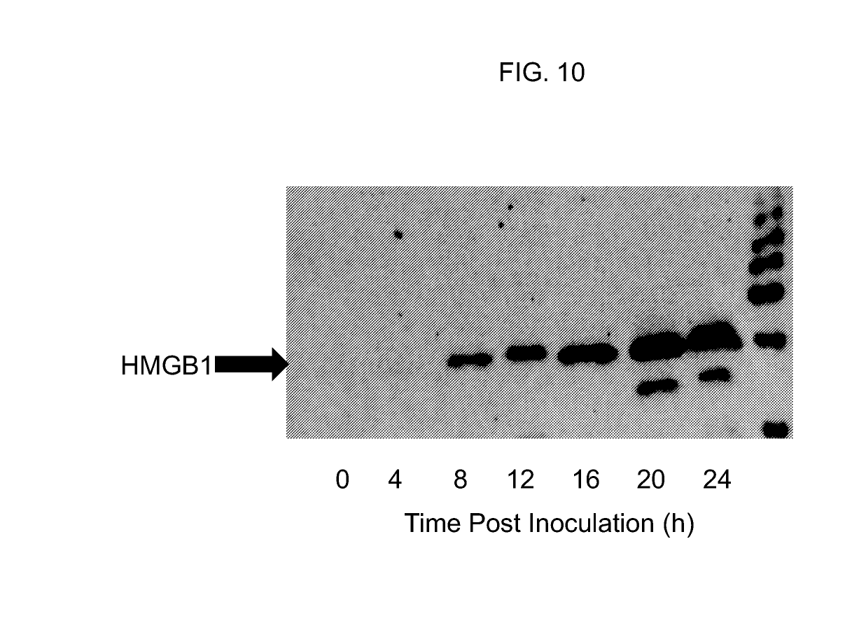

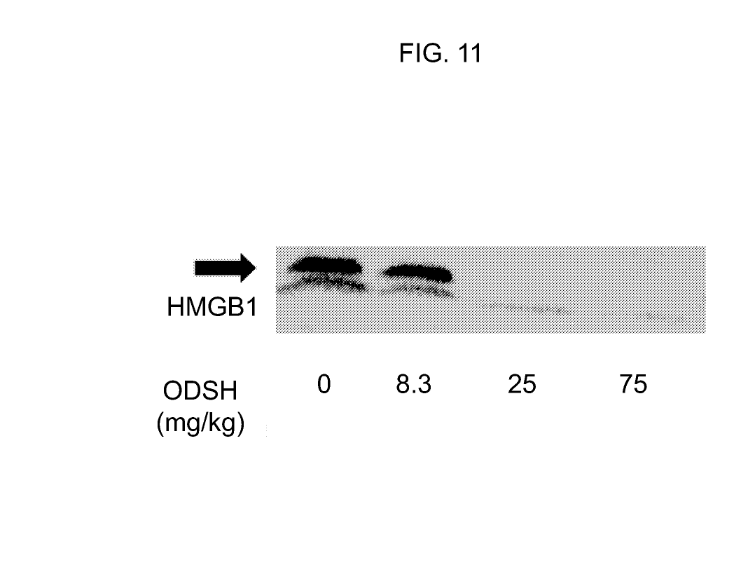

FIG. 10 shows a time-dependent increase in airway levels of HMGB1 in untreated P. aeruginosa-infected mice. In contrast, mice treated with 25 and 75 mg/kg of ODSH had markedly reduced airway HMGB 1 levels compared to mice treated with saline, as seen in FIG. 11,while ODSH at 8.3 mg/kg had no observable effect on HMGB1 levels. Following the inoculation and treatment regimen described in Example 2, levels of extracellular HMGB1 in BAL obtained every 4 hr up to 24 hr post-inoculation were measured using a competitive ELISA assay.

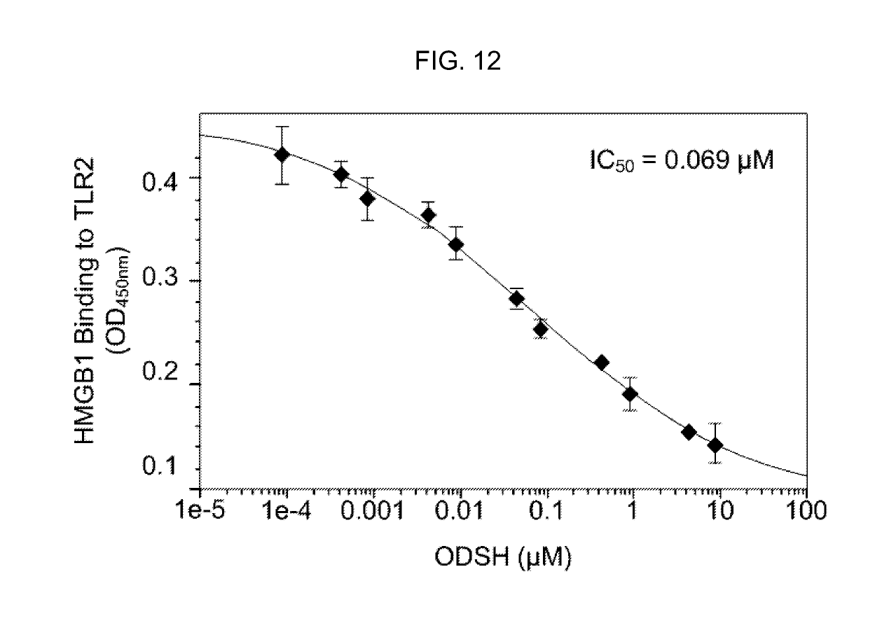

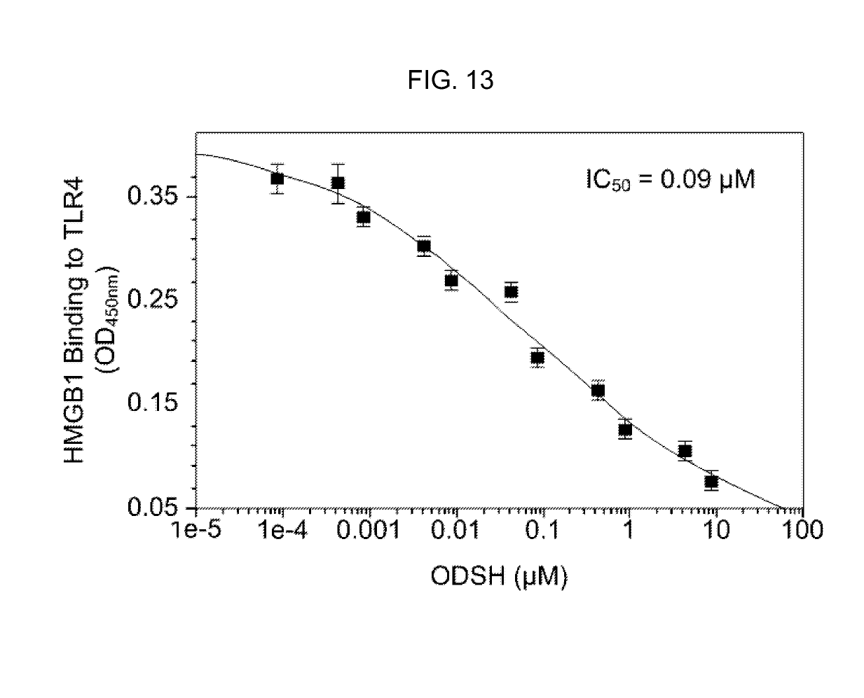

FIGS. 12 and 13 illustrate that increasing ODSH concentration result in decreased HMGB 1 binding to both TLR2 and TLR4, as indicated by reduced optical density. The concentration of ODSH that could reduce binding of HMGB1 to TLR2 and TLR4 to 50% of maximum level (IC₅₀) was 0.069 and 0.09 μM, respectively. To shows that only 25% of mice treated with saline survives at 48 hr while 50% of those treated with ODSH survived to this time. In addition to improved survival, mice in the ODSH group appeared more active compared to mice given saline that showed severe signs of illness including lethargy, huddling, and unwillingness to move upon stimulation.

### Example 7

### ODSH Improve Survival of Mice with P. aeruginosa Pneumonia

This experiment demonstrates that ODSH improved survival in mice inoculated with P. aeruginosa.

7.1 Materials & Methods

To ascertain the potential for ODSH to impact on host mortality/morbidity from P. aeruginosa infection, certain mice were anesthetized with intraperitoneal sodium pentobarbital. Thereafter, with a 1- to 2-cm incision, the trachea of each mouse was dissected and then inoculated with 0.5×10⁸ CFU of PAO1. P. aeruginosa was intratracheally inoculated to induce more severe injury and significant lethality in mice in order to determine the effect of ODSH on host survival. Mice were then administered 75 mg/kg ODSH or saline (control) subcutaneously ever 12 hr, starting at the time of inoculation. All mice were observed for up to 48 hr post-inoculation for indices of morbidity or for mortality and none of the mice received antibiotics.

To ascertain the potential for ODSH to impact on host mortality/morbidity from P. aeruginosa infection, certain mice were anesthetized with intraperitoneal sodium pentobarbital. Thereafter, with a 1- to 2-cm incision, the trachea of each mouse was dissected and then inoculated with 0.5×10⁸ CFU of PAO1. P. aeruginosa was intratracheally inoculated to induce more severe injury and significant lethality in mice in order to determine the effect of ODSH on host survival. Mice were then administered 75 mg/kg ODSH or saline (control) subcutaneously ever 12 hr, starting at the time of inoculation. All mice were observed for us to 48 hr post-inoculation for indices of morbidity or for mortality and none of the mice received antibiotics.

7.2 Results

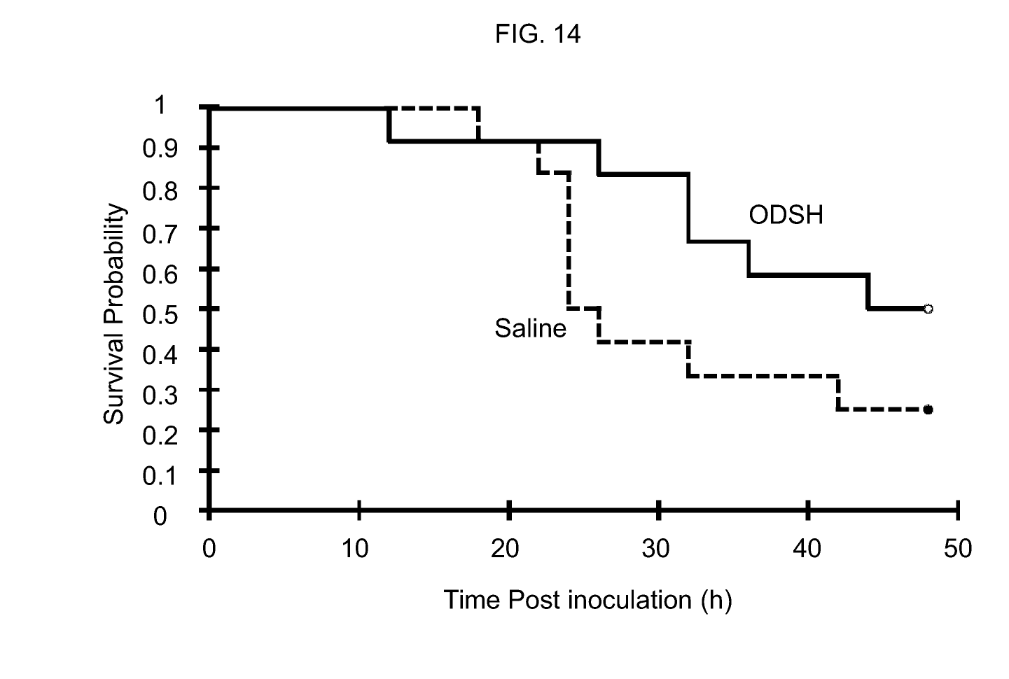

FIG. 14 shows that only 25% of mice treated with saline survives at 48 hr while 50% of those treated with ODSH survived to this time. In addition to improved survival, mice in the ODSH group appeared more active compared to mice given saline that showed severe signs of illness including lethargy, huddling, and unwillingness to move upon stimulation.

### 6. INCORPORATION BY REFERENCE AND NON-LIMITING DISCLOSURE

All publications, patents, patent applications and other documents cited in this application are hereby incorporated by reference in their entireties for all purposes to the same extent as if each individual publication, patent, patent application or other document were individually indicated to be incorporated by reference for all purposes.

While various specific embodiments have been illustrated and described, it will be appreciated that various changes can be made without departing from the spirit and scope of the invention(s).

1. A method of treating a bacterial lung infection, comprising:
administering a therapeutically effective amount of O-desulfated heparin (ODSH) to a subject suffering from a bacterial lung infection.

2. The method of claim 1, wherein the infecting bacteria are Gram positive bacteria.

3. The method of claim 1, wherein the infecting bacteria are Gram negative bacteria.

4. The method of claim 1, wherein the bacterial lung infection is a Pseudomonas infection.

5. The method of claim 4, wherein the Pseudomonas infection is chronic.

6. The method of claim 5, wherein the subject is suffering from cystic fibrosis.

7. The method of claim 6, wherein ODSH is administered parenterally.

8. The method of claim 7, wherein ODSH is administered subcutaneously.

9. The method of claim 7, wherein ODSH is administered intravenously.

10. The method of claim 6, wherein ODSH is administered by inhalation.

11. The method of claim 5, wherein ODSH is administered adjunctively to a second therapeutic agent.

12. The method of claim 11, wherein the second therapeutic agent is selected from the group consisting of: (a) an anti-microbial agent, (b) a DNase, (c) a bronchodilator, (d) a mucolytic agent, and combinations thereof.

13. The method of claim 12, wherein ODSH and the second therapeutic agent are administered via the same route.

14-19. (canceled)

20. The method of claim or 12, wherein ODSH and the second therapeutic agent are administered via different routes.

21-43. (canceled)

44. The method of claim 4, wherein the Pseudomonas infection is acute.

45. The method of claim 44, wherein the subject is hospitalized.

46. The method of claim 45, wherein the subject is intubated.

47. The method of claim 46, wherein the subject is on a ventilator.

48-65. (canceled)

66. A method of preventing a pulmonary Pseudomonas infection comprising:
administering an effective amount of O-desulfated heparin (ODSH) to a subject at risk for a pulmonary Pseudomonas infection.

67. A method of improving lung function in a subject suffering from cystic fibrosis, comprising:
administering to said subject a therapeutically effective amount of ODSH and dornase alfa.

68. A pharmaceutical composition comprising ODSH, a DNase, and a pharmaceutically acceptable carrier, diluent, and/or excipient.

69-71. (canceled)

72. A pharmaceutical composition comprising ODSH, an anti-microbial agent, and a pharmaceutically acceptable carrier, diluent, and/or excipient.

73. The pharmaceutical composition of claim 72, wherein the anti-microbial agent is selected from tobramycin, aztreonam, ciprofloxacin, and levofloxacin.

74. The pharmaceutical composition of claim 73, which is suitable for inhalation.

75. A unit dosage form comprising ODSH and a DNase.

76. The unit dosage form of claim 75, which is suitable for inhalation.

77-87. (canceled)

88. A kit comprising ODSH and a DNase, formulated for administration via the same route.

89. The kit of claim 88, wherein ODSH and the DNase are formulated for administration via inhalation.

90. The kit of claim 89, wherein ODSH and the DNase are formulated for inhalation using the same device.

91-92. (canceled)
